(19) United States
(12) Patent Application Publication
(10) Pub. No.: US 2017/0189033 A1
(43) Pub. Date: Jul. 6, 2017

(54) Occlusive Embolic Coil

(71) Applicant: MicroVention, Inc., Tustin, CA (US)

(72) Inventors: Ivan Sepetka, Los Altos, CA (US); Cathy Lei, Chino Hills, CA (US); Shannon Maguire, Allston, MA (US); Wendy Graczyk, San Clemente, CA (US); Jessica Liang, Irvine, CA (US); Hideo Morita, Irvine, CA (US)

(73) Assignee: MicroVention, Inc., Tustin, CA (US)

(21) Appl. No.: 15/399,635

(22) Filed: Jan. 5, 2017

Related U.S. Application Data
(60) Provisional application No. 62/275,692, filed on Jan. 6, 2016.

(51) Int. Cl. A61B 17/12; A61B 17/00

(52) U.S. Cl. CPC A61B 17/12113; A61B 17/0057; A61B 17/12031; A61B 17/1214; A61B 2017/00867

(57) ABSTRACT

An embolic coil is described used for occlusive purposes in the vasculature is described. The embolic or occlusive coil utilizes different techniques to both enhance the occlusive effect of the coil within the vasculature and to help guide the embolic coil into its deployed shape in the vasculature.

### RELATED APPLICATIONS

This application claims benefit of and priority to U.S. Provisional Application Ser. No. 62/275,692 filed Jan. 6, 2016 entitled Occlusive Embolic Coil, which is hereby incorporated herein by reference in its entirety.

### BACKGROUND OF THE INVENTION

Embolic material, such as coils, are typically used for occlusive purposes within the vasculature to treat issues such as aneurysms, arterio-venous malformations, arterio-venous fistulas, patent ductus arteriosis, left atrial appendages, fallopian type occlusion, and tumors. The embolic coils fill or occlude the target space, promoting clotting and preventing blood flow to the target region.

Completely filling the space of the target area is frequently a challenge. Embolic coils typically have an elongated delivery shape and a bunched delivered shape, the bunched delivered shape helps fill the space of the target region, but may not completely fill the target area. Portions of the coil can also overlap with each other to fill the space, however, this can still leave substantial open space. This open space is not desirable since it can potentially leave an open flow path for blood to still enter the target area, which negatively impacts clotting potential.

Various techniques can be used to augment the filling capabilities of embolic coils —including decreasing the size of the coil to augment the coil packing effect, introducing fibrous elements into or onto the coil to enhance the thrombogenicity and space filling potential of the coil, utilizing an expansile hydrogel material to augment the filling potential of an embolic coil, and/or utilizing an electropositive coil material in order to attract negatively-charged blood particles and thereby promote occlusion.

### SUMMARY OF THE INVENTION

Embolic or occlusive coils designed to enhance the space filling potential of the coils are described.

In one embodiment an ultra-thin injectable coil is described. The injectable coil can utilize an electropositive material, such as tantalum, to attract blood constituent particles in order to augment the thrombogenicity of the embolic coil. The injectable coil can utilize a number of shapes and designs in order to promote occlusion. The injectable coil can include thrombogenic enhancing agents, such as fibers, to further promote occlusion. The injectable coil can utilize an expansile agent such as hydrogel to augment the occlusive effect of the coil. In one embodiment, the injectable coil utilizes hydrogel coated fibers.

In one embodiment a pushable coil is described. In some embodiments, the pushable coil can be made of an electropositive material, such as tantalum, to attract blood constituent particles in order to augment the thrombogenicity of the embolic coil. Tantalum can be difficult to wind into a coil due to its material properties. In one embodiment, a tantalum coil utilizes break sections to create some open gap and some closed gap sections; the break sections promote the filling effect of the coil when the coil contacts a vessel wall. In some embodiments, a tantalum coil utilizes an inner member such as, for example, a wire, helical coil, cable, braid, or hypotube wound within the coil, the inner member has a high shape memory and helps to impart a coiled shape to the tantalum. In one embodiment, a tantalum coil utilizes a distal loop to promote a stacking effect for subsequent sections of the coil; in one embodiments both proximal and distal loops are use; in some embodiments, an inner member sitting within the tantalum coil has these proximal and/or distal loops. In one embodiment, the tantalum coil can include thrombogenic enhancing agents, such as fibers, to further promote occlusion. In one embodiment, the coil utilizes hydrogel coated fibers.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 illustrates an open pitch coil according to an embodiment of the present invention.

FIG. 2 illustrates a coil comprising closely wound sections and stretched sections according to another embodiment of the present invention.

FIG. 3 illustrates a coil comprising coil segments and suture segment according to yet another embodiment of the present invention.

FIG. 4 illustrates a coil comprising coil segments and chain segments according to yet another embodiment of the present invention.

FIG. 5 illustrates a coil comprising enlarged elements and links forming a chain according to yet another embodiment of the present invention.

FIG. 6 illustrates a coil comprising beads and links forming a chain according to yet another embodiment of the present invention.

FIG. 7 illustrates a coil comprising enlarged elements and sutures which is an alternative embodiment of FIG. 5.

FIG. 8 illustrates a coil comprising beads and sutures which is an alternative embodiment of FIG. 6.

FIG. 9 illustrates a coil comprising beads and hydrogel which is yet another embodiment of FIG. 6.

FIG. 10 illustrates a coil utilizing fibers according to an embodiment of the present invention.

FIGS. 11-12 illustrate a coil utilizing hydrogel and fibers according to another embodiment of the present invention.

FIGS. 13-14 illustrate a coil utilizing hydrogel segments and fibers according to yet another embodiment of the present invention.

FIG. 15 illustrates a coil utilizing a hydrogel middle section, and metallic elements at the proximal and distal ends according to yet another embodiment of the present invention.

FIGS. 16-20 illustrate a coil utilizing an inner member according to another embodiment of the present invention.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

Occlusive or embolic coils are used to treat a variety of vascular conditions, such as aneurysms. The occlusive or embolic coils are placed into the aneurysm or target site, filling the target site, cutting off blood flow to the region and promoting clotting over time to reduce the risk of rupture.

Coils typically adopt a primary or elongated configuration during delivery, that is a stretched configuration when constrained by the delivery catheter—and a secondary coiled configuration after delivery where the coil adopts its natural coiled shape when not constrained by the delivery catheter. Pushable coils are generally manipulated by the user via a pusher element, to push the coils through the catheter and into the treatment site. Injectable coils are very thin and thus are injected since pushing such a small coil through the catheter is difficult since the coils would likely bunch up within the catheter if pushed. Injecting the coils is a much quicker procedure and allows the coils to quickly navigate the delivery catheter and proceed into the vascular treatment site.

Many endovascular occlusive or embolic coils are constructed from platinum, which is considered an electronegative element according to the Pauling scale of electronegativity. Platinum also has a fairly high atomic weight and is radiopaque, properties that are useful for imaging coils in vivo. Blood constituent particles are generally electronegative. An electropositive material promotes the formation of thrombus when used intravascularly by attracting blood particles—thus augmenting the occlusive effect of the coils. Radiopaque materials that have a high atomic weight are also preferable in order to aid in visualizing the coils in vivo.

One particular material that meets all these criteria is tantalum, which has a lower electronegativity value on the Pauling scale than Platinum and is therefore more electropositive than Platinum. Tantalum also has a relatively high atomic weight which is useful for imaging purpose. Though tantalum is one specific material described, other materials with similar material properties (e.g., electropositive materials which are radiopaque and have a high molecular weight) could also be used with the embodiments described herein.

In one preferred embodiment, the electropositive implant is an injectable implant. Injectable coils are also called liquid coils since the coils are so thin that they do not retain their shape during delivery and thus must be injected with the help of a syringe instead of being physically pushed through a delivery catheter. One liquid coil is described in U.S. Pat. No. 5,669,931 which is hereby incorporated by reference in its entirety. Injectable coils are useful for occlusion, particularly for occluding small blood vessels, since they are thin and can therefore occlude a smaller space. These coils are also useful in occluding smaller vessels which may be too small for traditional coils, applications include occluding smaller vessels in the neurovasculature or smaller aneurysms in the neurovasculature, occluding feeder vessels to a tumor, occluding an AVM (arterio-venous malformations), or occluding fistulas, among other vascular conditions. Injectable coils are also beneficial for general occlusive purposes since they can be packed densely due to the small profile of the coil. The use of an electropositive injectable implant would combine the advantages of a dense occlusive formation while also allowing the coil to attract thrombus due to the electropositive nature of the coil itself to enhance the filling potential of the coil. In one example, an injectable coil is pre-loaded in an introducer tube. The introducer has a proximal and distal end. The proximal end connects to a syringe and the distal end is connected to a catheter. A syringe is connected to the proximal end of the introducer tube; when the syringe plunger is depressed, the liquid housed in the syringe (e.g., saline) is expelled and pushes the embolic coil through the catheter and into the vasculature.

In another embodiment the electropositive implant is pushable. The use of a pushable implant is possible where the coil is larger and can retain its elongated delivery shape in a delivery catheter. The use of a pushable implant is also feasible where a smaller catheter is used for delivery so that the coil keeps its elongated shape during delivery. The pushable implant would be delivered via a delivery pusher which the user manually grips and pushes.

The embolization coil embodiments described herein can be used for neurological or peripheral vasculature applications. Coils used in the neurovasculature region for occlusion are often smaller than coils used elsewhere in the vasculature since the blood vessels in the neurovasculature are smaller than elsewhere in the body. Therefore, it might make sense to use an injectable coil for neurovasculature applications while using a pushable coil for peripheral regions. However, various embodiments are contemplated. For example, the coil embodiments described herein can be injectable for neurovasculature use and pushable for peripheral vasculature use. Alternatively, the coil embodiments can be injectable for both neurovasculature and peripheral vasculature use. Alternatively still, the coil embodiments can be pushable for both neurovasculature and peripheral vasculature use.

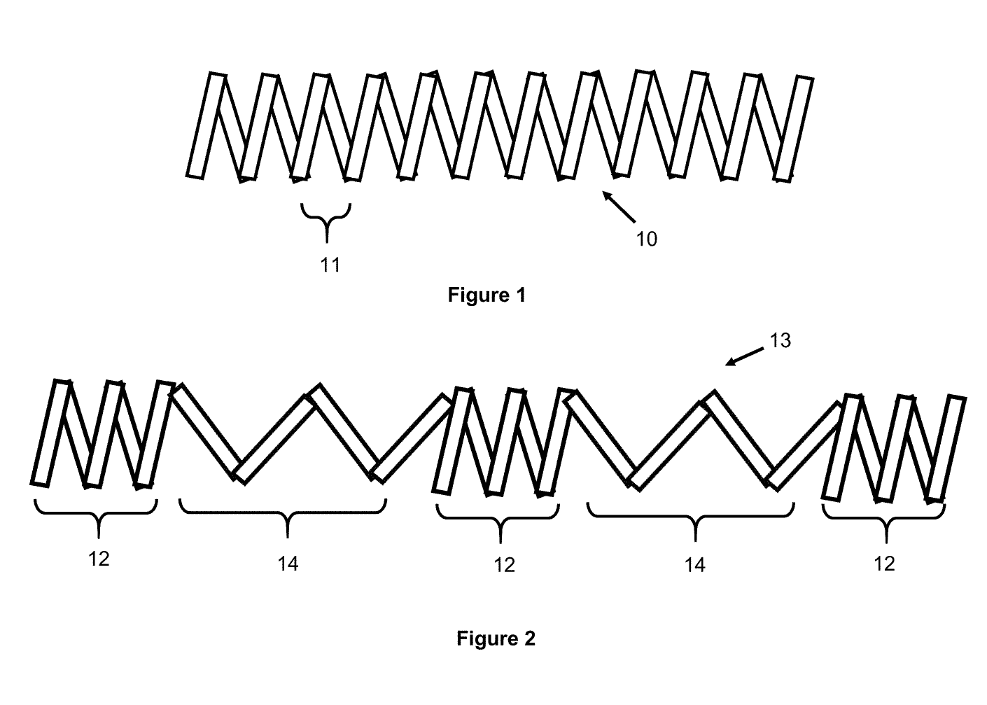

FIGS. 1-4 show various coil shape embodiments. The coils have particularly beneficial use as an injectable coil or as an injectable electropositive (e.g., tantalum) coil, but can also be used as a pushable coil. A first embodiment of a coil 10 as shown in FIG. 1 is an open pitch coil (meaning there are gaps 11 between subsequent windings, instead of a closed pitch where there is no gap between windings) with a consistent shape. In one example, the coil may have a diameter of about 0.0005″ to about 0.00015″ for use in the neurovasculature and have a diameter of about 0.0015″ to about 0.003″ where used in the peripheral vasculature. In another embodiment, a closed pitch coil can be used where the sequential coil windings are nested together.

Another embodiment of a coil 13 is shown in FIG. 2. The coil is wound with a variable pitch where there is a closer wound section 12 utilizing closely spaced windings alternating with a stretched section 14 where the windings are not closely spaced. Such a configuration is useful to promote more of a folding/kinking effect where the coil is used to fill part of the vasculature, such as an aneurysm. The diameter profile of the coil is similar to the one described in FIG. 1. The length of the closer wound section 12 can be about 0.5 cm to about 5 cm, whereas the length of the stretched section 14 can be about 0.05 cm to about 0.2 cm. Generally, the closer wound sections 12 and stretched sections 14 will alternate with each other, however, the length of the sections can be customized to promote less or more folding in various regions of the coil. Thus close coil section 12 will usually be followed by stretched coil section 14 and this sequence can continue throughout the coil. However, the length of closer wound coil section 12 in one part of the coil may be different than the length of closer coil section 12 in another part of the coil. Similarly, the various stretched coil sections 14 may have different lengths. Though FIG. 2 shows three close wound sections 12 and two stretched sections 14, this is just shown as an illustrative example and various combinations of close wound sections/stretched sections are possible.

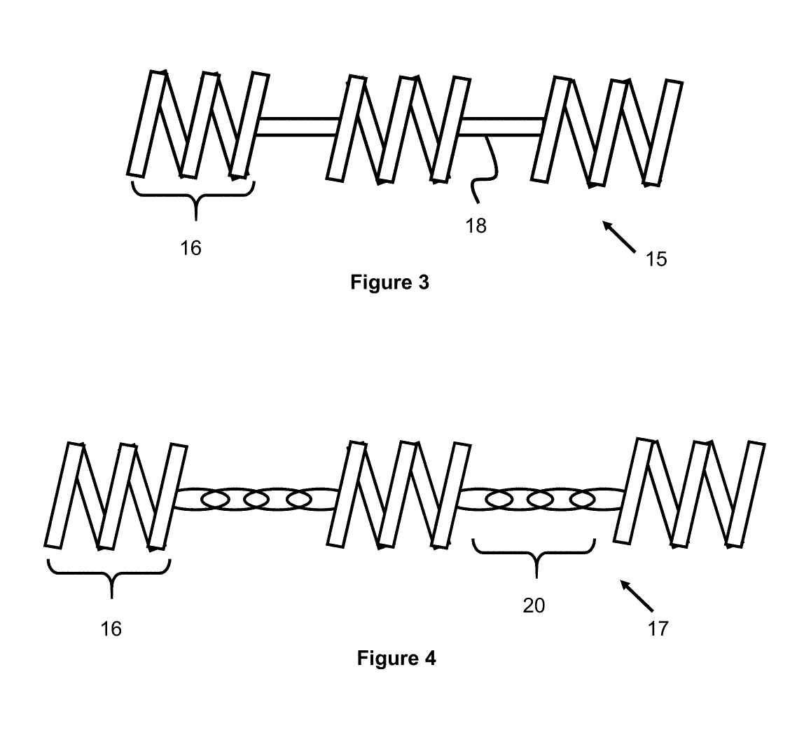

FIG. 3 shows another coil 15 embodiment comprising a series of open pitch coils segments 16 with suture segments 18 in-between. The suture can be composed of a polymer, a thin metal, or combinations therein. In one example each coil segment is about 0.5-5 cm and the suture length is about 0.02″-0.1″.

FIG. 4 shows another coil 17 embodiment comprising a series of open pitch coil segments 16 with chain segments 20 in-between. In one example the coil segments are about 0.5-5 cm in length. The chain segments can be a series of mechanical links linked together, analogous to jewelry chain links.

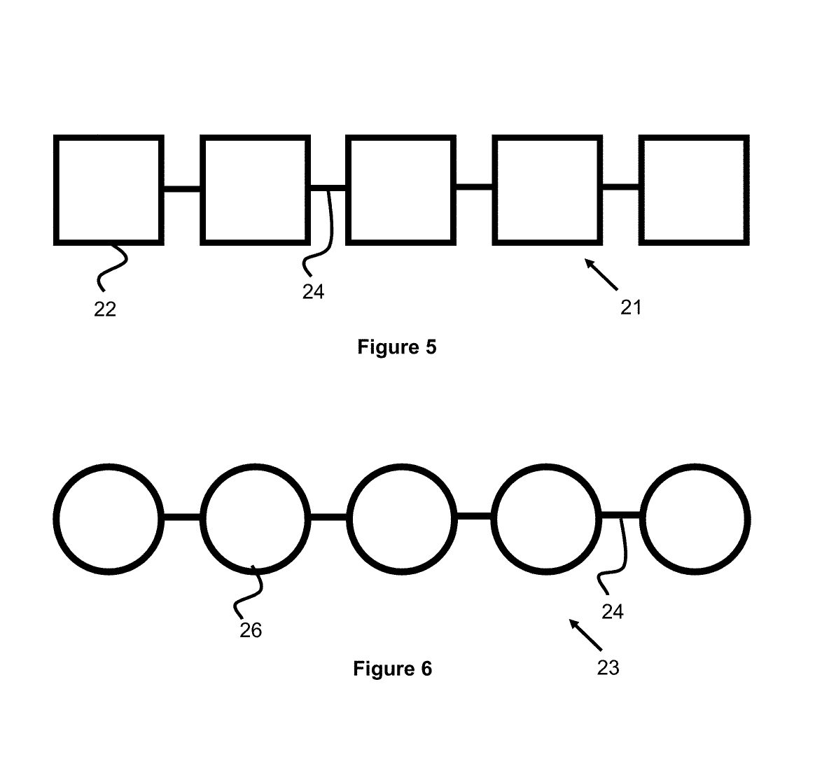

FIGS. 5-9 illustrate various coil chain embodiments utilizing chain-like links, where the chains are created by a series of linked elements.

FIG. 5 shows a chain embodiment 21 analogous to a jewelry chain comprising a series of enlarged elements 22 connected by a series of links 24. The elements 22 are referred to as “enlarged elements” to demarcate them from the smaller links. In one example the enlarged elements have an outer diameter of about 0.005″-0.30″ and length of about 0.01″-0.05″ and the links have a length of about 0.01″-0.05″. The enlarged elements can take on a number of shapes, (a non-inclusive list includes square, cylindrical, rectangular, spherical, ovular, and combinations therein). The enlarged elements are comprised of an electropositive material such as tantalum and the links can also be comprised of tantalum, or another metal—such as nitinol or stainless steel.

FIG. 6 shows a chain embodiment 23 similar to the one of FIG. 5, with the only distinction being the inclusion of a series of spherical beads 26 in place of the enlarged elements 22 of FIG. 5. In one example the beads have a diameter of about 0.005″-0.03″ and the links have a length of about 0.003″-0.01″. The beads are comprised of an electropositive material such as tantalum.

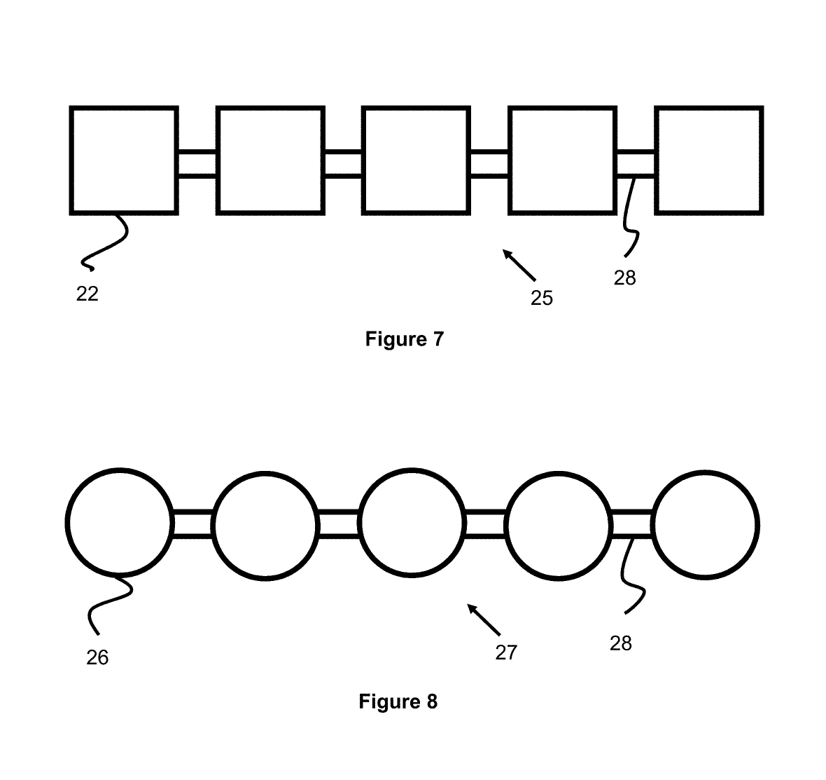

FIG. 7 shows a chain embodiment 25 comprising a series of enlarged elements 22 similar to the enlarged elements of FIG. 5, connected together via sutures 28. In one example the sutures can be composed of a polymer, such as Dacron. The enlarged elements 22 can be cylindrical, square, or rectangular in shape. Where the enlarged elements are cylindrical, the elements can have a length of about 0.005″-0.05″ and a diameter of about 0.005″-0.03″. The enlarged elements are comprised of an electropositive material such as tantalum.

FIG. 8 shows another chain embodiment 27. The embodiment is similar to the one of FIG. 7 except spheres or beads are utilized instead of the illustrated rectangular enlarged elements 22 shown in FIG. 7.

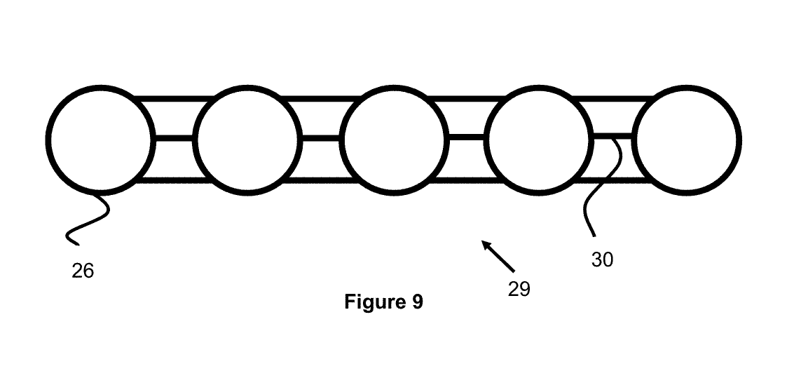

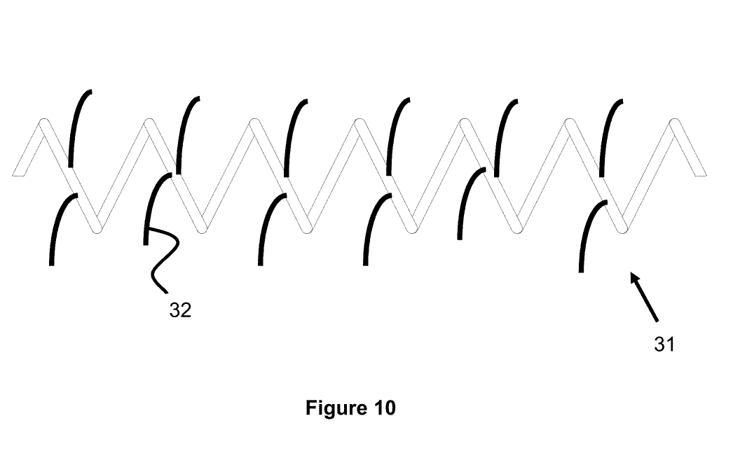

FIG. 9 shows another chain embodiment 29 comprising a series of beads 26 having hydrogel links 30 between the beads 26. Hydrogels are materials that swell or expand when exposed to a substance of a certain pH; when used for vascular applications, hydrogels are made to expand when exposed to blood, based on the pH of blood. The hydrogel links expand on contact with blood thereby increasing the radial profile and, therefore, the space filling effect, of the links and the chain once in the vasculature. In one example, the link could be a metallic material (e.g., a metal cable or thread) coated with hydrogel. In another example the link 30 itself is made of hydrogel. In one example the bead 26 diameter is about 0.005″ to 0.03″ and the hydrogel link length 30 is about 0.005″-0.05″. In one example, the amount of hydrogel is tailored so that the links expand to a diameter close to the diameter of the beads to create a substantially consistent diameter for the embolic chain once it's within the vasculature. Though beads are shown in FIG. 9, numerous shapes can be used including cylindrical, square, rectangular, etc.

In another embodiment, the enlarged elements or beads of the implants shown in FIGS. 1-9 may be selectively coated with hydrogel in order to enhance the space filling potential of the implant. Some or all of the elements/beads may be coated; the links connecting the elements/beads together may also be coated with hydrogel.

The embodiments shown in FIGS. 3-9 generally utilize an enlarged member (e.g., element 16 of FIGS. 3-4 or elements 22 and 26 of FIGS. 5-8) and a smaller member between the enlarged members (e.g., elements 18, 20 of FIGS. 3-4, element 24 of FIGS. 5-6, element 28 of FIGS. 7-8). One advantage of this configuration is that the smaller regions provide a nesting section where other portions of the coil or other coils can fill the space between the enlarged and smaller members, thereby augmenting the occlusive effect of the coil.

Several of the implant embodiments described herein utilize an electropositive material such as tantalum in order to attract blood particles and thus increase the filling potential of the implants. Some implant embodiments are injected as a “liquid coil” due to the small size of the implant. The implants may also be scaled up in size to make them pushable, or can be delivered through a smaller microcatheter to enable pushing as a method of delivery. The use of a smaller microcatheter would eliminate the issue of the smaller implants not retaining their primary, elongated shape during delivery which otherwise would cause deliverability issues. In one example where a pushable delivery system is used, a mechanical, thermal, or electrolytic detachment system can be used to separate the implant from the pusher upon correct placement within the vasculature. US2010/0268204 and US2015/0289879 disclose thermal detachment systems which can be used and are hereby incorporated by reference in their entirety.

As discussed before, liquid or injectable coils may be used for a number of reasons and offer a number of advantages as compared to conventional pushable coils. As described earlier, they can be more densely packed than traditional pushed coils due to their small size and thus offer some occlusive space filling advantages. These coils are also useful in occluding smaller vessels which may be too small for traditional coils. Thus the coils may be useful to occlude feeder vessels to a tumor, or occluding blood flow through AVM (arterio-venous malformations), or fistulas, among other vascular conditions.

The method of using injectable coils will now be described. A saline syringe can be used to flush the catheter line. The microcatheter is guided and placed appropriately within a vasculature, the injectable coil is then injected through another syringe through the microcatheter and is deployed at the target location.

In one embodiment the injectable coil is pre-loaded in an introducer and is placed proximal to the catheter hub. The proximal end of the introducer is then connected to a syringe filled with saline. Depressing the saline plunger will expel the saline and push the injectable coil through the microcatheter to deploy the coil within the vasculature.

Various other embodiments are also possible for injectable coil delivery. For example, the coil can be pre-loaded or pre-placed into the syringe and the introducer tube can be avoided entirely. In other embodiments, a material besides saline can be loaded into the syringe and used to deliver the coil.

Several alternative configurations are also possible in order to produce an electropositive pushable or injectable coil. In one embodiment, a nitinol or stainless steel coil is used, but the coil can be coated with an electropositive material (e.g., tantalum). In another embodiment a coil is coated with hydrogel and the hydrogel layer is coated with an electropositive (e.g., tantalum) layer. This electropositive layer may have gaps in order to allow the hydrogel to expand. The space filling potential of the coil is augmented by the hydrogel filling as well as the electro-positivity of the material attracting more blood particles. In another embodiment, the coil itself is made of an electropositive material such as tantalum, and the coil is coated with a noncontiguous layer of hydrogel. The hydrogel will expand on contact with blood to enhance the occlusive, space-filling effect of the coil; meanwhile, areas of the coil without the hydrogel layer will utilize an electropositive material which attracts constituent blood particles, augmenting the occlusive nature of the coil even in the regions of the coil not utilizing a hydrogel.

In another embodiment, the saline or other liquid material which is used to inject the coil may contain some electropositive material, for example tantalum flakes or tantalum powder, to impart an electropositive effect onto the coil. In one example, the saline can have particles of an electropositive material floating within it. The injectable coil could include recesses or grooves to trap the electropositive material, thereby imparting an electropositive property on portions of the coil.

Please note, the embodiments presented above and herein could be used with injectable or pushable coils. Either coil will adopt an elongated configuration during delivery and a coiled configuration when released from the delivery device. In the case of an injectable coil, the pressure applied to the injectable coil via the syringe and the liquid delivery medium (e.g., saline) as well as possibly the size of the delivery catheter will keep the injectable coil in an elongated shape when tracked through the catheter, while the injectable coil will adopt a bunched or coiled configuration when placed within the vasculature. With injectable coils, the goal is to create a homogenous mass of small coil elements which congeal together to form a firm occlusive structure. For pushable coils, the restraining force provided by the catheter maintains the coil in its elongated form as the coil is pushed through the catheter. The coils are heat set to naturally adopt a secondary shape-memory infused shape, and the coils will adopt this secondary shape once freed from the delivery catheter and deployed within the vasculature. Like injectable coils, the goal is to create a homogenous mass of coiled elements which congeal together to form a firm occlusive structure. The difference is that injectable coils may be preferable to pushable coil for particular scenarios, such as smaller treatment sites.

Other previously discussed embodiments utilized different constituent elements of the coil sized differently than other elements of the coil (e.g., the embodiments of FIGS. 3-8 which utilize smaller and larger regions). The larger regions of the coil would take up more volumetric space than the smaller regions of the coil, although the smaller regions of the coil would provide a nesting area for other coil sections. Other embodiments could utilize different sized regions but still allow for a relatively consistent shape profile after delivery. For instance, a larger sized element of the coil chain could contain less hydrogel or a smaller electropositive coating, whereas a smaller sized element of the coil chain could contain more hydrogel or more of an electropositive coating. Different regions of the coil would have different sizes, but the post-delivery profile of the coil would be similar after expansion or the hydrogel and/or thrombus formation on the coil itself “evened out” the overall size of the coil.

The following embodiments utilize embolic coils that include fibers in one or more locations along the coil. These fibers enhance the thrombogenic nature of the coil by enhancing the space filling and occlusive effect of the coil. Some of these embodiments are injectable, with a method of delivery similar to the other injectable coil embodiments described earlier, and some embodiments are pushable. In some embodiments, a metallic coil could include nitinol or even a radiopaque material such as platinum to aid in visualization. Alternatively, tantalum could be used; tantalum, as discussed earlier, is both radiopaque and electropositive which aids in imaging as well as thrombus formation.

The use of fibers with embolic coils has several advantages. Often, coil sections will overlap each other but still leave void spaces which are too small to fill. The fibers can fill these void spaces. Another advantage is that, due to the thrombogenic nature of the fiber itself, the fiber will attract blood particulates and thereby speed up the time it takes to occlude the target site.

FIG. 10 shows a picture of a coil 31 utilizing fibers 32 in one or more locations throughout the coil. The fibers may be tied to the external surface of the coil or adhesively affixed to the coil. The fibers, as previously described, offer some advantages including promoting thrombogenesis and occlusion. While the fibers can take on any range of sizes, one advantage of using fibers is that they can be thinly sized which is beneficial for filling the small spaces of a target site (e.g., an aneurysm) where the coil diameter may ordinarily be too large to completely occlude the target space. The thrombogenic nature of the fibers also enhance the occlusive effect of the coil. The fibers can comprise various materials, for example various polymers such as (but not limited to) PTFE, PET, Nylon, Dacron. In one embodiment, the fibers are coated with hydrogel to further augment the occlusive effect of the fibers.

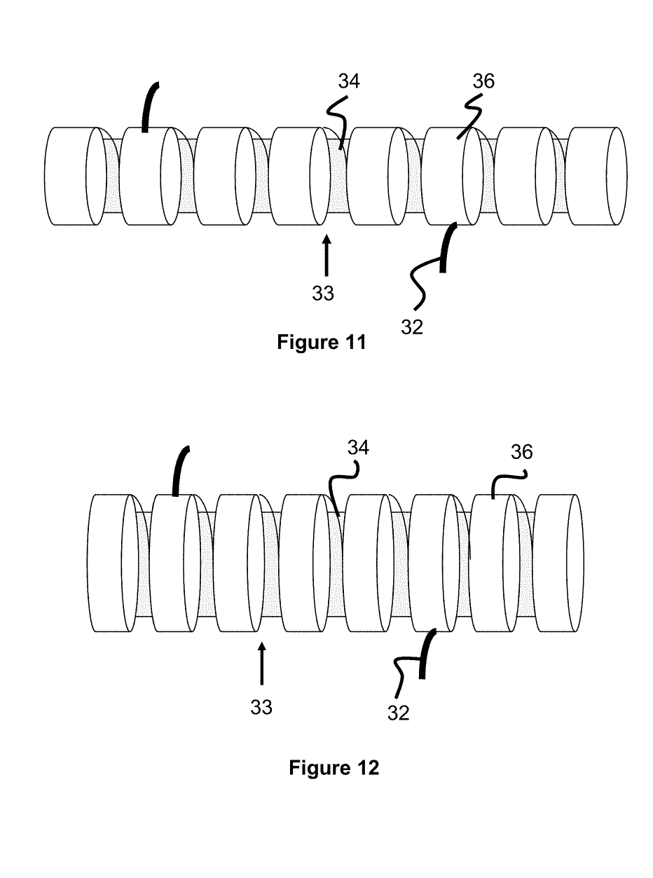

In another embodiment 33, hydrogel 34 can be placed within the windings of the coil 36. The hydrogel 34 can be stretched to a predefined length wherein the outer diameter of the hydrogel will shrink; this allows the hydrogel string 34 to fit within the interior space of the coil 36. FIGS. 11-12 show this arrangement where the coil 33 contains hydrogel 34 placed within the winding 36 of the coil. The fibers 32 may be fixed in one or more locations along the coil 33. In one example, the fibers 32 are placed on the winding of the coil itself (e.g., via mechanical tying or adhesive). In another example, the fiber 32 could be placed on or around the hydrogel. FIG. 11 shows the coil 33 before expansion of the hydrogel 34, while FIG. 12 shows the coil 33 after the hydrogel 34 expands.

As the hydrogel 34 is exposed to blood, the hydrogel 34 expands and the hydrogel expansion exerts pressure on the coil windings which, in turn, also causes the coil's overall diameter to increase. In another embodiment, segments of hydrogel 34 are selectively placed throughout the coil 33. While FIGS. 11-12 illustrate one piece of hydrogel 34 placed within the lumen of the coil 36, this alternative approach would utilize various segments of hydrogel placed throughout the coil embodiment 35. These hydrogel segments may all be sized the same, or may be sized differently. Some segments of the coil would include hydrogel and other segments would not—as shown in FIG. 13 where regions 34a and 34b have hydrogel but the region in between does not. The fibers 32 could be placed in regions of the coil devoid of the hydrogel, or, alternatively, the fibers 32 could be placed throughout the coil.

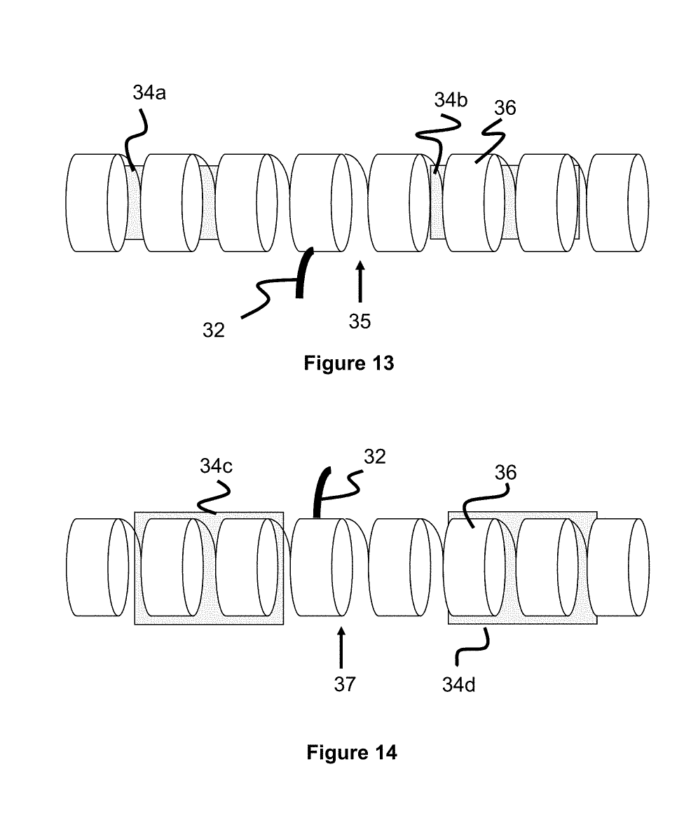

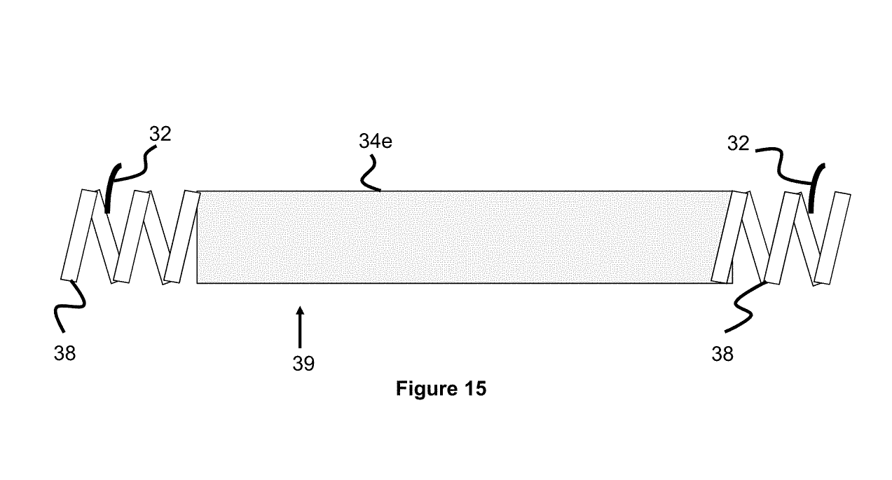

FIG. 14 shows another embodiment 37 in which hydrogel 34 and fibers 32 are used, however, here the hydrogel 34 is placed over the coil 36 in various locations throughout the coil embodiment 37 as regions 34c and 34d, rather than within the lumen of the coil 36, as in the embodiment 35 of FIG. 13. The fibers 32 may be placed in regions where there is no hydrogel, which would enhance the occlusive effect of the coil sections where there is no hydrogel present. However other embodiments could utilize the fibers in various locations throughout the coil 36 including in locations where there is hydrogel already utilized. For these embodiments, the hydrogel could either be placed over the coil or the coil could be placed within the hydrogel.

FIG. 15 shows another embodiment 39 of a coil 38 utilizing hydrogel 34. This embodiment utilizes a solid hydrogel cylinder 34e spanning the majority of the length of the embolic coil 38, and having proximal and distal metallic coiled loops 38 are attached at each end of the hydrogel cylinder 34e. To manufacture this design, the hydrogel cylinder 34e is produced and the proximal and distal coil elements 38 are then affixed to either end of the hydrogel cylinder 34e. In this regard, the only metallic coil elements 38 are those at the proximal and distal ends of the implant 39, while the rest of the implant 39 solely comprises a solid hydrogel with no actual metallic coil under or over the hydrogel cylinder 34e. Fibers 32 may then be placed on the proximal and distal loops 38. Alternatively, fibers may also be placed throughout the length of the hydrogel cylinder 34e. Alternatively, fibers 32 could only be placed on the hydrogel cylinder 34e but not on the proximal and distal loops 38. With this embodiment, the coiled end loops 38 may provide relatively sturdy terminal ends for the implant 34e, while the hydrogel material expands when exposed to blood in vivo and takes up most of the space within the target region. The use of thrombogenic fibers 32 may further enhance the occlusive potential of the implant 39.

Another embodiment utilizes a coil and a piece of hydrogel placed over the majority of the coil, leaving the proximal and distal ends of the coil free. Visually, this would look similar to the embodiment of FIG. 15, except that it would include a coil under the hydrogel layer, with only the proximal and distal ends of the coil being hydrogel-free.

Another embodiment would utilize a coil and a piece of hydrogel placed under the majority of the coil, leaving the proximal and distal ends of the coil free. The space between adjacent windings of the coil could be controlled so that, for instance, a large gap between adjacent windings would still allow the hydrogel to expand past the diameter of the coil since the retention force restraining the hydrogel would be minimal. Alternatively, if it was desirable to keep the expanded hydrogel within the coil or to otherwise minimize hydrogel expansion, the gap between adjacent coil windings would be kept small to provide a higher retention force restraining the hydrogel.

The fibered embolic coils of FIGS. 10-15 could be injected in some embodiments, or pushable in other embodiments. A pushable version of the various coils shown could adopt a complex or three-dimensional shape. Complex coils are typically larger coils that adopt a complex three-dimensional shape upon delivery—the coils are wound and heat-set over a mandrel into a complex or three-dimensional shape, and the coil adopts this three-dimensional shape naturally when not restrained by the delivery catheter. These complex coils are typically useful as framing coils to frame the periphery of the treatment site (e.g., an aneurysm), although complex coil shapes may also be used with filling coils to fill the treatment site (e.g., aneurysm) in particular applications. Complex coils are discussed in U.S. Pat. No. 8,066,036, U.S. Pat. No. 9,089,405, and US20120041464 all of which are hereby incorporated by reference in their entirety. Injectable coils, pushable coils, and methods of using and delivering both types of coils were described in detail earlier; the same principles for use and delivery would apply either injectable or pushable versions of the fibered coils shown and described in FIGS. 10-15.

Fibers could also be utilized on the coil designs shown and described in FIGS. 1-9. Fibers could be located throughout the coil or placed in one or more selective locations along the length of the coil.

Previous discussion in the specification mentioned the use of electropositive material in creating an injectable or pushable embolic coil, where the inclusion of an electropositive material would offer some occlusive benefits since the material would attract oppositely-charged blood constituent material. Tantalum was mentioned as one particularly beneficial electropositive metal which could be used in a coil due to its electropositivity and radiopacity. Though tantalum would offer some positive effects if used as a coil material, tantalum can be a difficult material to work with. A coil contains two shapes—primary, or elongated delivered shape (its ‘straight’ shape when being delivered through a catheter) and a secondary shape (its ‘coiled’ shape upon delivery). The typical heat and shape setting processes used to impart the secondary shape in typical metallic coil materials, such as platinum, are difficult with tantalum. The typical shape and heat setting process involves winding the coil on a mandrel to impart its coiled shape, and then heat-setting the coil to impart the coiled shape-memory, such that the coil will naturally adopt its secondary coiled shape upon delivery. Tantalum tends to become brittle once it's heated after being wound into its secondary shape which makes the shape setting operation difficult. Tantalum also oxidizes at a lower temperature than platinum which also makes the heat setting operations difficult.

The following embodiments address these issues to create a usable tantalum embolic coil. Please note, these embodiments could also be used for materials besides tantalum which are also difficult to work with. For example, these techniques and designs could be used on other materials which are similar to tantalum in that they have are electropositive, and have high molecular weight and strong radiopacity. Fibers can optionally be placed in or more locations throughout the coil in the following embodiments to further augment the thrombogenic nature of the coil. These embodiments could also be used with traditionally used shape-memory materials (e.g., nitinol) typically used to create coils, in order to mitigate some of the stiffness issues that would otherwise associated with creating very large-diameter coils, to thereby create flexible large-diameter complex coils.

One way to address some of the material issues with tantalum is to create a design which makes it more likely that the coil will turn in a particular direction and not become stuck against the wall of the blood vessel, without necessarily utilizing a large number of loops. It can be difficult to place a large number of loops on a tantalum coil since the loop-making procedure involves winding the coil over a mandrel and heat setting the coil This is a challenge given the material qualities of tantalum discussed earlier, including how easily it can oxidize and become brittle. Various breaks can be introduced into the coil design so that the coil would have a series of looped sections as well as a series of open or “break” sections without loops.

Please note, for terminology a loop or closed loop will refer to a looped section, while “open,” “open loop,” “break,” and similar terms will refer to a non-looped section. In one example, a loop can be followed by an open section, which is followed by a loop, which is followed by an open section, which is followed by a loop, etc. In another example, the loop/open sections alternate based on segment length; thus a certain length of the coil will have loops, a certain length will have breaks or open/un-looped sections, and this proceeds in an alternating arrangement. Even if an open or break section of the coil contacts a vessel wall, the coil will naturally want to turn inwards due to the presence of the loops on either side of the open section. Thus, less loops are required (a high number of loops being a manufacturing challenge), while the coil will still assert a coiled shape due to the inherent shape memory imparted by the adjoining looped sections next to the open sections. This design can be visualized in FIG. 2, which shows this design with an earlier embodiment (in the earlier portion of the discussion, this design was described as utilizing a stretched and close-wound section, which is essentially the same idea here). Alternatively, this design would visually look similar to FIG. 2, except stretched section 14 would be straight and horizontal, where section 14 alternates with close-wound section 12. The proximal and distal ends of the coil are preferably looped so that the rest of the coil can stack against those end loops.

Building off the proximal and distal end loop concept, given the tantalum material properties discussed above, it may be difficult to create end loops on a tantalum coil. Yet, end loops are desirable first so that a distal loop contacts the vessel wall first in order to provide a soft surface for the rest of the coil to fill against—and next so that subsequently deployed coils can contact a softer coiled element to minimize resistance during occlusive packing utilizing several embolic coils. In one embodiment, an inner nitinol wire is wound through the entirely of the coil (conceptually, this should be thought of as an inner wire sitting within the coil)—the proximal and distal ends of the coil are cut so only the nitinol wire is exposed at the ends. The wire ends are pre-wound to impart shape memory onto the wire ends so that the proximal and distal ends of the wire naturally coil. The proximal and distal loops solely comprise the nitinol wire, while the rest of the coil comprises the tantalum coil which the nitinol wire sits within. Fibers can also optionally be placed in one or more locations throughout the coil to augment the thrombogenicity of the embolic coil. The advantage of this design is that the looped ends are provided by the nitinol wire instead of the tantalum coil, which mitigates the problem associated with creating looped shapes out of tantalum.

Another way to address the material challenges of tantalum is to add a helical shape memory material, such as nitinol, PEEK, or MP35N into the inner diameter of the coil. These materials are easier to manufacture than tantalum and it is much easier to create a coiled heat set shape from these materials. The operating principle is to create a helical shape memory structure which sits within the tantalum coil, the presence of a helical shape memory structure within the tantalum coil will help impart a helical shape memory into the tantalum coil itself. The inner helical shape memory alloy material will coil causing the outer tantalum material to also coil. In this way, the tantalum material is guided into a coiled or complex shape by the inner shape memory material.

Here, the tantalum coil has a primary shape when being delivered through the catheter, this primary shape can be thought of as a delivery or elongated shape. The inner material placed within the tantalum coil also has a delivery or elongated shape during catheter delivery. The tantalum coil has a secondary shape when freed of the delivery catheter, this secondary shape can be thought of as a coiled or deployed shape. The inner material also has a coiled or deployed shape when unconstrained by the delivery catheter. The inner shape-memory material naturally wants to adopt its deployed shape when freed from the delivery catheter and this, in turn, guides the tantalum coil into its secondary or deployed shape.

In one embodiment, a single nitinol wire 40 is used within the inner diameter of the coil 41. The nitinol wire can have a size range of about 0.00195″-0.004″. The wire can be wound over a mandrel and heat set for 10 minutes at 500 degrees Celsius to impart its secondary, coiled shape. The nitinol wire is inserted into the inner diameter of the tantalum coil, the tantalum coil and nitinol wire are secured at the proximal and distal ends using adhesive, such as UV glue. An additional glue ball can be externally added to both ends of the device as well. Additional adhesive can also be used throughout the length of the wire, between the tantalum coil and the nitinol wire in order to enhance the connectivity between said nitinol wire and said tantalum coil. Fibers can optionally be added along the length of the tantalum coil; in one example, the fibers are spaced equally over the length of the coil, in another example the fibers are spaced in a more random manner. In other embodiments, multiple nitinol wires can be used or a thin nitinol hypotube can be used. Other materials can also be used such as PEEK or MP35N.

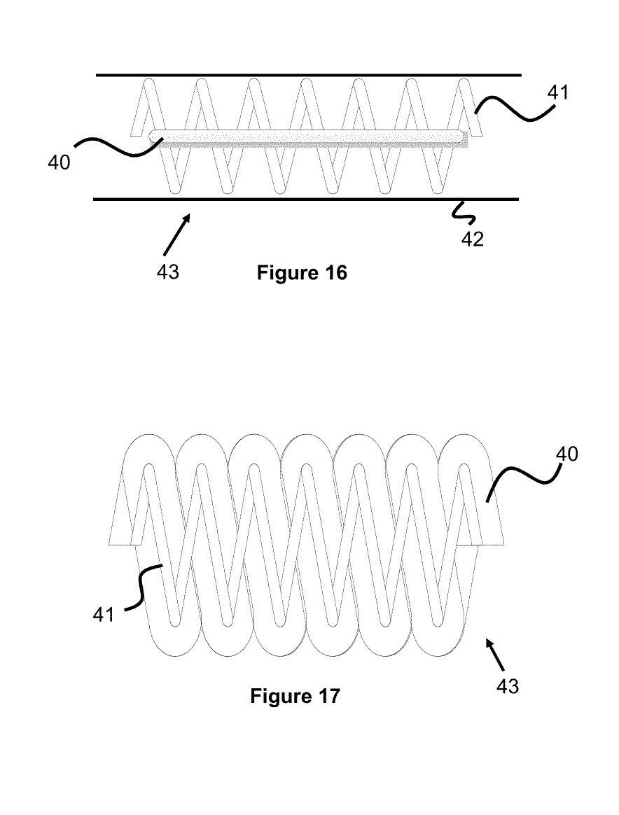

This design is shown in FIG. 16, where there is a tantalum coil 41 and inner member 40. The inner member 40, as discussed earlier, can be a wire, coil, hypotube, multiple wires, or multiple wires formed into a braid or cable—basically any shape that can easily adopt a looped or curved shape memory. The tantalum coil 41 initially takes its primary, elongated configuration when pushed through a delivery catheter 42 as shown in FIG. 16. The tantalum coil's primary, elongate shape still contains sequential windings defining a lumen as shown in FIG. 16, and inner member 40 sits through the lumen defined by the sequential windings.

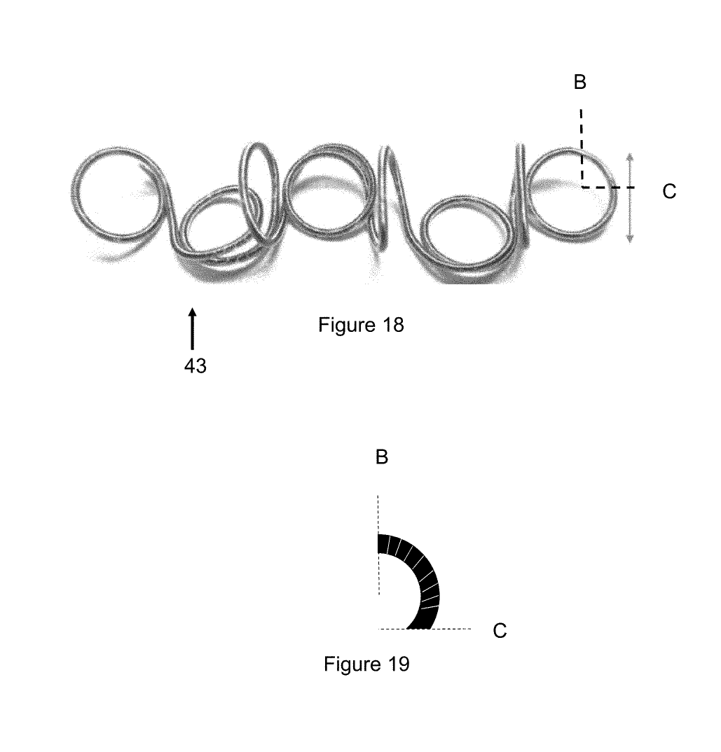

In FIG. 17, tantalum coil 41 is in its secondary, coiled shape which it adopts when it is not constrained by the catheter 42. FIG. 17 can be appreciated in the context of FIGS. 18-19; FIG. 18 shows an example of a complex coil shape which, in this example, utilizes a series of loops. The tantalum coil implant adopts this shape when not constrained by the delivery catheter, so this image represents the secondary shape of the coil. FIG. 19 represents the cross-sectional view of a quarter of one of the loops of FIG. 18, taken between lines B-C of FIG. 18 and shows a number of coil windings nested next to each other. FIG. 17, therefore, represents the portion of the secondary shape of the tantalum coil shown in FIG. 19. When tantalum coil 41 is pushed from the delivery catheter and deployed in the vasculature, the inner member 40 adopts its secondary or coiled shape. As the inner member adopts its secondary shape, the inner member will cause the tantalum coil 41 to also adopt its secondary, coiled shape since the inner member is affixed to the inner diameter of tantalum coil 41. Therefore, inner member 40 guides the outer tantalum 10 into its secondary, coiled shape.

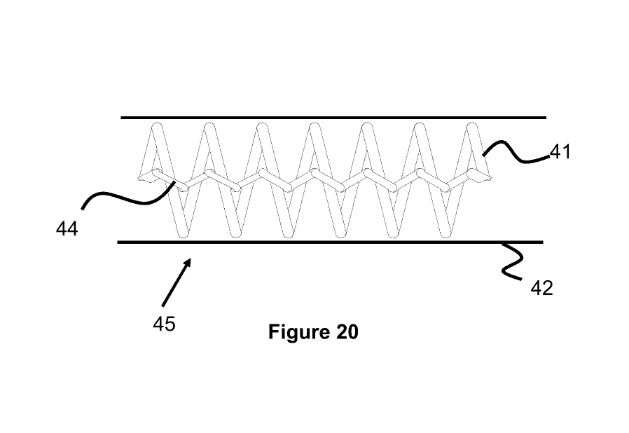

In another embodiment 45, a nitinol coil is used within the inner diameter of the tantalum coil. Instead of a wire which is subsequently wound into a coiled shape for its secondary shape, a nitinol coil 44 is used as shown in FIG. 20. The nitinol coil 44 is then subsequently wound onto a mandrel to impart a different coiled secondary shape. Thus the primary (elongate, delivery) shape is akin to a stretched coil with windings present (e.g., as shown in FIG. 20), while the secondary shape is an un-stretched coil—similar to the example of FIG. 17. One advantage of using a coil 44 as an inner member as opposed to a wire is that a stretched coil is biased to return to its coiled shape. Therefore, using a coil 44 as an inner member within the outer tantalum coil 48 will provide increased impetus for the outer tantalum coil to adopt a coiled shape as the inner coil also adopts its secondary coiled shape.

The manufacturing operation is similar to the inner nitinol wire procedure described above. The initial shape of the nitinol inner member is a coiled shape, the coil can be wound over a mandrel and heat set for 10 minutes at 500 degrees Celsius to impart its secondary, coiled shape. The nitinol coil is inserted into the inner diameter of the tantalum coil, the tantalum coil and nitinol coil are secured at both ends using adhesive, such as UV glue. An additional glue ball can be used to further secure both coil elements to each other, and fibers can optionally be placed throughout the tantalum coil. In other embodiments, multiple nitinol coils can be used. In other embodiments, the one or more nitinol inner coils can be affixed to the outer tantalum coil throughout the length of both coils. Other materials can also be used such as PEEK or MP35N. The manufacturing technique for different materials may change since each material has different shape memory properties. Thus MP35N would be heat set at 850 degrees Celsius for 30 minutes, while PEEK would be heat set at 280 degrees Celsius for 10 minutes.

In another embodiment, a braided cable wire is used for inner member 40 instead of a wire or coil. Multiple nitinol wires with a diameter of about 0.0013″-about 0.0015″ are braided or twisted and annealed to form a cable wire. The cable wire is wound on a mandrel and heat set at 500 degrees Celsius for ten minutes to create the desired, coiled secondary shape. The cable wire is inserted into the inner diameter of the tantalum coil contouring the primary wind tantalum to its shape, similar to what is shown in FIG. 16 (except inner member 40 is a braided cable wire instead of the single wire shown). Glue or adhesive can be used to secure the elements to each other, and fibers can optionally be placed around the outer diameter of the tantalum coil.

In another embodiment, inner element 40 comprises multiple single nitinol wires which are braided into a cylindrical, tubular shape. As opposed to the braided cable wire embodiment earlier, the cylindrical tubular shape would have a hollow interior with walls formed by the braided wires. The cylindrical, tubular shape is heat set into a secondary helical shape by wrapping the braid around a mandrel. The heat treated braid is inserted into the inner diameter of the tantalum coil (as shown in FIG. 16), giving the coil its secondary shape (as shown in FIG. 17). UV glue is used to lock the cylindrical unit to the tantalum coil, and distal balls are used as well as described earlier. Fibers are optionally placed around the tantalum coil.

The use of the inner element 40 within the tantalum coil 41 in the preceding embodiments to impart a secondary shape into the tantalum coil 41 may make it unnecessary to actually wind the tantalum coil 41 over a mandrel to impart a secondary shape within the tantalum coil itself. In other words, the natural shape memory of the inner member 40 may be sufficient to impart a coiled secondary shape into the tantalum coil 41 without having to heat-set the tantalum coil 41 at all to imprint its secondary shape. However, this will mostly depend on a number of variables, including the shape memory of the inner member itself, and the size of both the inner member and the outer tantalum coil. In other words, in various embodiments described utilizing inner member 40, the tantalum coil 41 may not need to be wound over a mandrel and heat-set into a secondary shape at all since the inner member will provide enough of a kick to guide the outer tantalum coil 41 into its secondary shape. In various embodiments, the tantalum coil may still need to be wound over a mandrel and heat set into a secondary shape, however, the heat setting can be done at a lower temperature or over a shorter amount of time than would otherwise be needed since the inner member will help guide the tantalum coil into its secondary shape. This would mitigate many of the workability issues associated with tantalum when it is exposed to heat treatment to impart shape memory.

Please note though the preceding embodiments discussed methods of helping to impart a secondary shape on a tantalum coil, given how difficult tantalum can be to manufacture, these techniques can be used on various embolic coil materials such as platinum, stainless steel, cobalt-chromium, nitinol, etc.

Please note figures shown are meant only as representations and/or illustrations to aid in understanding, and not limited to what is explicitly shown. Similarly, any measurements are meant only as illustrative examples to aid in understanding and are not meant to be limited to what is explicitly stated.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. An embolic device comprising:
a tantalum coil with a primary shape and a secondary shape;
the tantalum coil fitted with an inner shape-memory material to assist in forming the secondary shape;
the inner-shape memory material separately adopting a delivery shape when constrained within a catheter and a deployed shape when freed from the catheter;
wherein the tantalum coil adopts the shape of the inner shape-memory material, such that the tantalum coil adopts the delivery shape when constrained within the catheter as the primary shape and the deployed shape when freed from the catheter as the secondary shape.

2. The embolic device of claim 1, wherein the inner shape-memory material is made of nitinol.

3. The embolic device of claim 1, wherein the inner-shape memory material is a wire, a cable, a braid, a coil, or a hypotube.

4. The embolic device of claim 1, further comprising fibers placed along the length of the coil.

5. The embolic device of claim 1, wherein the tantalum embolic coil adopts a complex, three-dimensional shape in its deployed shape.

6. An injectable embolic device comprising:
a plurality of radially enlarged elements and a plurality of radially reduced elements arranged in an alternating manner, wherein the enlarged elements are comprised of tantalum.

7. The injectable embolic device of claim 6, wherein the radially reduced elements provide a nesting region for other embolic devices deployed in the vasculature.

8. The injectable embolic device of claim 6, wherein the plurality of radially reduced elements include hydrogel.

9. The injectable embolic device of claim 6, wherein some sections of the device are made of nitinol.

10. The injectable embolic device of claim 6, where the radially reduced elements are sutures.

11. The injectable embolic device of claim 6, where each radially reduced element is a linked chain segment.

12. The injectable embolic device of claim 6, further comprising fibers placed along the length of the device.

13. An embolic device comprising:
a tantalum coil;
an inner element within the tantalum coil, the inner element having proximal and distal looped ends;
where the proximal and distal looped ends of the inner element sit respectively past the proximal and distal ends of said tantalum coil.

14. The embolic device of claim 13, further comprising fibers placed along the length of the coil.

15. The embolic device of claim 13, wherein the proximal and distal ends of the tantalum coil are cut so that the proximal and distal looped ends of the inner element are exposed.
